(19) United States
(12) Patent Application Publication
(10) Pub. No.: US 2021/0169777 A1
(43) Pub. Date: Jun. 10, 2021

(54) Artificial Saliva with Electrolytes for Xerostomia

(71) Applicant: Chun Nan Lin, Rosemead, CA (US)

(72) Inventor: Chun Nan Lin, Rosemead, CA (US)

(21) Appl. No.: 17/106,132

(22) Filed: Nov. 29, 2020

Related U.S. Application Data
(60) Provisional application No. 62/944,236, filed on Dec. 5, 2019.

(51) Int. Cl. A61K 9/00; A61K 9/06; A61K 8/04; A61Q 11/00; A61K 8/21; A61K 33/06; A61K 8/19; A61K 8/24; A61K 33/42; A61K 33/20; A61K 8/20; A61K 31/047; A61K 8/34; A61K 8/73; A61K 47/38

(52) U.S. Cl. CPC A61K 9/006; A61K 2800/48; A61K 8/042; A61Q 11/00; A61K 8/21; A61K 33/06; A61K 8/19; A61K 8/24; A61K 33/42; A61K 33/20; A61K 8/20; A61K 31/047; A61K 8/345; A61K 8/731; A61K 47/38; A61K 2800/92; A61K 9/06

(57) ABSTRACT

The present invention provides an orally ingestible composition comprising electrolytes, Methyl Hydroxybenzoate, and sorbitol for treating xerostomia (dry mouth) and associated disorders, with very good results in provide a protective coating inside the mouth and throat, in alleviating the symptoms of xerostomia, including difficulties in eating and speaking, the pain associated to xerostomia, improving the quality of life on affected people as well. Sweeteners, colorants, and preservatives also combined in the artificial saliva. Methyl Hydroxybenzoate acts as a lubricant and as a delivery vehicle for water from the composition to moisten the mouth. The artificial saliva composition is used to keep moisture in the mouth when there is little or no natural saliva is produced, providing relief from dry mouth, as a symptom of xerostomia, mucositis, and stomatitis, as well as general inflammation and/or ulceration of mucous membranes, while avoiding pH imbalances which may lead secondary to tooth decay and/or injury to the upper digestive tract including the oral cavity, oropharynx, or esophagus. The pH buffering system includes acidic and basic components. The electrolytes include potassium, sodium, magnesium, calcium, chloride and phosphate together with fluoride. The composition of the invention has also been proved in clinically to prevent demineralization from acid conditions and decreasing risk of oral thrush. The composition of the invention can apply by persons with certain diseases, such as some cancers, radiation, chemotherapy, old age, smoking, and both prescribed and over the counter medications with side effect.

### CROSS-REFERENCE TO RELATED APPLICATION

This application is a continuation priority to and the benefit of U.S. Patent Provisional Application Ser. No. 62/944,236 filed on Dec. 5, 2019 with titled “Composition and Methods for relief of oral and throat discomfort”, the entire disclosure of which is hereby incorporated reference.

### BACKGROUND OF THE INVENTION

### Description

Xerostomia, also known as dry mouth, is a condition in which lacking of saliva or the amount or quality of saliva diminishes within the oral cavity and throat take place and cause multiple problems due to insufficient salivary production. Xerostomia has affected millions of people in the world which can lead to numerous health, nutritional, and social hazards. Xerostomia is not a disease itself, but is a universal problem associated with several medical conditions, such as side effects of a variety of prescribed and over the counter medications, old age, smoking radiation, and chemotherapy. Some common complains associated with xerostomia include constant sore throat, burning sensations, difficulty speaking, eating, and swallowing, itching throat, and dry nasal passages, all related to the decreased level of fluids in the oral cavity. Sore mouth and throat often developed dry area in the mouth and cause speech became strained and painful which can cause sufferers withdrawal from social contact and lead to disillusionment and depression. The consequences of dry mouth can affect the quality of life of anybody, by increasing the risk of caries, risk of oral thrush, and periodontal diseases, physical damage to mucous membranes in the mouth, throat, and esophagus, and also causing bad breath and bad taste to the mouth.

There are many commercial saliva substitute products available intended for treating xerostomia (dry mouth) in the market. However, those saliva substitutes cannot be fully satisfactory.

Xerostomia may be reduced by the drinking fluids, chewing gums, oral sprays, mouthwashes and lozenges. Artificial saliva substitutes can be also used as palliative treatments. There are some examples of artificial saliva include compositions which contain electrolytes that mimic those found in natural saliva, sodium carboxymethylcellulose-based preparations to provide the proper level of viscosity which similar to natural saliva.

Xerostomia is not a disease itself, but xerostomia is contributing factor for both minor and serious health problem which associated with diseases such as radiotherapy of the head and neck, chemotherapy, infection or dysfunction of the salivary glands, inflammation of the mouth and throat, taking medication (anticholinergic, sympathomimetic, antihypertensive groups, opioids, benzodiazepines, anti-migraine agents), anemia, depression, bulimia, genetic disorder, age, fever, emotional factors such as fear or anxiety, obstruction of the salivary ducts, diabetes, HIV, smoking, alcoholics, drug addicts, Bell's Palsy, and Sjogren's syndrome. Xerostomia is also liable for secondary effects, including an increased incidence of dental caries demineralization of teeth, and increased risk of secondary infections such as candidiasis which is the most common oral infections. Therefore, fluoride therapy often includes adjunctive treatment for xerostomia for caries control and antifungal medications such as nystatin. Therefore, dryness of the mouth, or xerostomia, is a common disease that affects a large number of the population transiently or permanently.

The xerostomia can cause symptoms of burning tongue, mouth, pharynx, and esophagus which are sensitive to spicy food and beverages. Sometimes individuals can affect speech and swallowing. It is difficult to treat caries and periodontitis because the reduce secretion of saliva results in more bacteria retention in the oral cavity and on the teeth. The individuals with xerostomia are associated with reducing of resistance of the mucosa against colonization of bacterial and fungal infection. When people wear removal prosthesis, often have a great problem with retention of the prosthesis and infection of the mucosa which is result in the dryness of the mouth.

Saliva is a complex mixture of fluids, protein, enzymes, and electrolytes that are produced by the salivary glands which surround the oral cavity, and is secreted into the mouth following appropriate stimulation of oral tissues. Secretion of saliva is regulated by the body's autonomic nervous system which permits involuntary secretion following an appropriate stimulus such as testing or chewing food, smiling or thinking of food, or having any object within the mouth which stimulates the oral mucosa.

The salivary glands of the mouth normally daily production around 1-1.5 liters of saliva and it must be utilized a saliva substitute when xerostomia appears which has to be kept in such a volume per 24 hours. Therefore, artificial saliva agent has to possess such retention and lubricating properties that it is retained and moistens the teeth and mucosa for a relatively long time after spray to the mouth and throat have been taken. Furthermore, the artificial saliva has to be swallowable so it will reduce the dry mouth symptoms in the pharynx and the esophagus. The artificial saliva composition has a similar to naturally saliva so both of them have very similar pharmacokinetics.

The present invention has a different approach to reducing xerostomia. The oral composition herein described a combination of electrolytes. The artificial saliva may be used to treat dryness of the mouth and throat (e.g. hyposalivation, xerostomia, mucositis), regardless of the cause and the condition is either temporary or permanent. The artificial saliva relieves dryness of the oral mucosa and/or decreased pain. The artificial saliva can be used for medical assessment and performed by health professional.

The artificial saliva may be topical oral administrated few times per day whenever patients need. The artificial saliva maybe used up to 4 times daily. Thus, the artificial may primarily be used at home, office, aircraft, or other non-clinical setting. Also, the artificial saliva can be used in clinical or healthcare facility setting wherever patients are receiving inpatient services.

The artificial saliva can be used as part of oral hygiene program for patients with xerostomia. The artificial saliva provides intensive hygiene for the oral cavity and protection of oral tissue, and may be used to help relieve bad taste, relieve offensive nasal discharge, facilitate speaking/eating, crusting, and offset the symptoms of xerostomia such as dental caries, remineralization of teeth, oral thrush, or inflammation of the mucous membrane.

The artificial saliva has low concentration of fluoride, so it is not unlikely to occur intoxication or overdoses even when high dose of artificial saliva are administrated clinically.

The artificial saliva may also be used as an adjunct to standard oral care in treating the mucositis that may be caused by radiation or high dose chemotherapy.

When spray the artificial saliva onto tongue or toward the back of the mouth then swallow, the artificial saliva can forming film for 4-5 hours for keeping moisture. The artificial saliva is formulated by electrolytes such as Sodium, Magnesium, Calcium, Phosphate, Potassium, Chloride, and Fluoride, which is manufactured to mimic the composition and action of saliva and a pH similar to the saliva with pH 6.75 to 7.0.

All percentages, parts and ratios are based upon the total weight of the artificial saliva composition of the present invention, unless otherwise specified.

This artificial saliva may contain an agent to increase viscosity, such as carboxymethylcellulose or hydroxyethylcellulose, minerals such as calcium and phosphate ions and fluoride, preservatives, and flavoring agents.

The artificial saliva has packaged in the PET bottle with 100 mL with spray applicator.

The artificial saliva may further comprise ingredients with an anti-inflammatory, an anti-microbial, and an antifungal. The anti-microbial ingredient can be Sodium Carboxymethylcellulose (CMC) and sorbitol. The antifungal agent can be methylparaben.

There are many agents against xerostomia on the market, but these are very varying as to efficiencies. Serious disadvantages in connection with the agents on the market are inter alia short term and minor effects, and in some cases with an unpleasant taste. Moreover, the artificial saliva prices are usually high in general, but most xerostomia persons have to utilize such agent continuously even though economic drawback.

According to the present invention, the artificial saliva are provided a good condition for eliminate or at least significantly reduce the disadvantages of the previously commercial agents and which fulfil the requirements and better set up on agents for the patients.

The artificial saliva is highly water-soluble, and mostly contained in the oral cavity and spread over the oral mucosa, gum, and teeth. The artificial saliva will provide electrolytes saturated solution of phosphate, calcium, and fluoride to the oral cavity. Some of the calcium, phosphate, and fluoride ions are incorporated with dentin of the teeth when remineralization occurs. Since the amount of artificial saliva is very low in the oral cavity, fluoride is unlikely to cause any adverse effects from intoxication.

It can also be added that it has been shown that high relative proportions of sodium carboxymethylcellulose (CMC) which gives a high viscosity and helps lubricate the oral cavity. So when the temperature increases, the viscosity decreases. When increase concentration of Sodium CMC, the viscosity increases, which has been found that it decreases the severity of oral dryness and the effect of oral dryness on daily life in D-J Oh study in 2008. He also concluded that CMC-based artificial saliva demonstrated moderate effects in reducing dry mouth-related symptoms and behaviors with more significant effects appearing in patients whose residual secretory potency was severely compromised In the case of sorbitol, because of its anti-caries effects in addition to providing sweetness, it is preferred to have a weight in the artificial saliva formulation of at least 4%, so as to be capable of protecting against tooth decay.

The preparations of artificial saliva have usually higher viscosity than water and similar to the viscosity of saliva.

In the artificial saliva composition, the individual components and their respective weight percent as Table 1 below.

| Ingredient | Weight Percent |
|---|---|
| Dipotassium hydrogen phosphate | 0.01-1 |
| Potassium dihydrogen phosphate | 0.02-1 |
| Potassium chloride | 0.05-0.8 |
| Magnesium chloride | 0.01-0.5 |
| Calcium chloride | 0.001-0.05 |
| Sodium fluoride | 0.001-0.05 |
| Sorbitol | 1-5 |

Additional components may be added to the artificial saliva for reducing inflammation, and/or acting as bactericidal, antiviral, and/or antifungal agent. The artificial saliva comprising at least one of an anti-inflammatory, an analgesic and/or anesthetic, an antimicrobial, and an antifungal. The artificial saliva composition for use in a human oral cavity which consisting of dipotassium hydrogen phosphate, potassium dihydrogen phosphate, potassium chloride, magnesium chloride, calcium chloride, sodium fluoride.

The artificial saliva comprises purified water.

In view of the entirety of the present disclosure, including the claims, a person having ordinary skill in the art will readily recognize that the present disclosure introduces a composition comprising artificial saliva for use in a human oral cavity, the artificial saliva comprising: dipotassium hydrogen phosphate; potassium dihydrogen phosphate; magnesium chloride; sodium fluoride; and calcium chloride.

The artificial saliva may comprise, by weight: between about 0.01% and about 1.0% of dipotassium hydrogen phosphate; between about 0.02% and about 1.0% potassium dihydrogen phosphate; between about 0.05% and about 0.8% of potassium chloride; between about 0.001% and about 0.05% of sodium fluoride; between about 0.01% and about 0.05% of magnesium chloride; between about 1.0% and about 5.0% of sorbitol; and between about 0.001% and about 0.05% of calcium chloride.

The composition may already comprise water, and the composition is electrolyte-like solution.

The composition may relieve symptoms of dryness of the oral cavity.

The present disclosure also introduces a composition consisting of artificial saliva for use in a human oral cavity, the artificial saliva consisting of: dipotassium hydrogen phosphate; potassium dihydrogen phosphate; magnesium chloride; sodium fluoride; and calcium chloride.

Incorporation of a thickening and binder agent into the man-made saliva fluid is effective to make the oral cavity moist and smooth and to retain the moisturizing effect. Nevertheless, when sodium carboxymethylcellulose formulated into a salivary formulation, resulted in the physical properties of the artificial saliva is similar to human saliva. In view of this, sodium fluoride is included for mineralization of the dentition (Alpoz etal. 2008). When increases viscosity with the increasing concentration of the artificial saliva formulated, and as a result, the resulting viscosity exerted its physical action to thereby provide the salivary preparation with the cushioning property and consequently the improved feeling of use.

### REFERENCES

Patents Cited in This Cited by

| | | Application | Publication |
|---|---|---|---|
| Title | Assignee | Date | Date |
| Dissolving powders | Invado | 2009 Oct. 26 | 2011 Apr. 14 |
| that can be mixed | Pharmaceuticals | | |
| with water and used | LLC | | |
| as an oral rinse | | | |
| Method of providing a | Burton Parsons | 1971 Jun. 21 | 1973 Oct. 23 |
| synthetic mucus in vivo | & Co Inc. | | |
| Oral lubricant for | The Board Of | 1983 Dec. 19 | 1985 Aug. 27 |
| athletic mouth | Regents, | | |
| protector | University Of | | |
| | Texas System | | |
| Oral hygiene | Firmenich SA | 2000 Jun. 21 | 2001 Oct. 23 |
| compositions which | | | |
| mask the burn | | | |
| sensation and the | | | |
| astringency of | | | |
| eucalyptol and zinc | | | |
| Formulations and | Invado | 2014 Jul. 24 | 2015 Jan. 29 |
| methods for treating | Laboratories, | | |
| oral inflammation, | LLC | | |
| injury, or pain | | | |
| Artificial saliva | Lion Corporation | 1986 Mar. 24 | 1989 Nov. 07 |
| composition | | | |
| Analgesic and anti- | Cambridge | 2005 Feb. 16 | 2006 Aug. 17 |
| inflammatory | Scientific | | |
| composition | PTY LTD | | |
| Formulations of | Valeant | 2015 Nov. 19 | 2016 Jun. 23 |
| calcium and phosphate | Pharmaceuticals | | |
| for oral inflammation | International, INC | | |
| Saliva substitute | Camurus Ab | 1991 Apr. 10 | 1993 Nov. 09 |
| Self-film-forming | AB-Biotics, S, A, | 2016 Jul. 11 | 2017 Jan. 26 |
| Composition for oral | | | |
| care | | | |
| Oral hygiene | Beecham group, | 1987 Apr. 03 | 1987 Dec. 30 |
| compositions | PLC. | | |
| Oral care compositions | Colgate- | 2017 Jun. 22 | 2017 Dec. 28 |
| | Palmolive | | |
| | Company | | |
| Oral care composition | Unilever N.V, | 2017 Apr. 13 | 2017 Nov. 23 |
| comprising tricalcium | Unilever PLC, | | |
| silicate | Conopco, INC | | |
| | D/B/A Unilever | | |
| Solid oral care | Unilever N.V, | 2016 Mar. 23 | 2016 Oct. 06 |
| Compositions | Unilever PLC, | | |
| | Conopco, INC | | |
| | D/B/A Unilever | | |
| Oral care compositions | Colgate- | 2016 Dec. 16 | 2017 Jun. 22 |
| comprising at least one | Palmolive | | |
| phosphate/acrylate | Company | | |
| copolymer and at least | | | |
| one cationic active | | | |
| ingredient | | | |
| Oral care compositions | Colgate- | 2015 Oct. 08 | 2017 Apr. 13 |
| and methods of using | Palmolive | | |
| the compositions | Company | | |
| Method of providing a | Burton Parsons & | 1971 Jun. 2 | 1973 Oct. 23 |
| synthetic mucus in vivo | Co Inc. | | |

1. An artificial saliva composition is a gel which contains in purified water: potassium chloride, sodium fluoride, magnesium chloride, and calcium chloride, characterized in that electrolytes: Sodium, Potassium, Chloride, Calcium, Phosphate, Magnesium and Fluoride with excipients which are Methylparaben, Sorbitol, sodium carboxymethylcellulose, flavor and colorant. All the proportions are less than 10% w/v and have a pH value adjust to between 6.75 and 7.

2. A composition according to claim 1, further comprising a sugar alcohol as an isotonic agent, and as a dental agent is sorbitol.

3. A composition according to claim 1, further comprising a sodium methylcellulose as a non-ionic thickening agent.

4. A composition according to claim 1, further comprising a sodium fluoride as a decay preventing compound and a dental agent for inhibiting growth of dental microorganisms.

5. The artificial saliva composition claim 1, wherein comprising a colorant for coloring the gel.

6. The artificial saliva composition claim 1, wherein comprising a flavoring compound for flavoring the gel.

7. A composition according to claim 1, wherein it contains one or more excipient agents which have antifungal, bactericidal, and antiviral properties.

8. A composition according to claim 1, for use in the treatment of saliva deficiency.

9. The method of claim 1, wherein the composition is sprayed into oral cavity.

10. The artificial saliva composition claim 3, wherein a neutralizer for setting a viscosity of the gel, adjusting a pH level of the gel.

11. The artificial saliva composition claim 3, wherein a preservative for preserving the gel, and the viscosity of the gel is similar to natural saliva and can be safely used orally.

12. The artificial saliva composition claim 1, wherein has a low near-liquid viscosity for allowing electrolytes, especially calcium, to travel through the matrix of the saliva substitute quickly enough to be help remineralization.

13. According to claim 1, wherein the artificial saliva is applied directly on a back of tongue of the mouth to be treated and spread thoroughly inside the mouth, and swallowed by the subject.

14. According to claim 1, wherein the artificial saliva is reapplied as desired whenever dry mouth of dry throat appears possibly four or more times per day.

15. According to claim 1, wherein the artificial saliva is provided with a spray applicator.

16. According to claim 1, wherein the artificial saliva is applied once for overnight use, without reapplication.

17. A bottle for relief dry mouth with artificial saliva is provided, the bottle comprising, the artificial saliva of claim 1, and instructions for use of the artificial saliva.
